(19) United States
(12) Patent Application Publication
(10) Pub. No.: US 2015/0343709 A1
(43) Pub. Date: Dec. 3, 2015

(54) DATABASE AND MARKETPLACE FOR MEDICAL DEVICES

(71) Applicant: OSIRIS BIOMED 3D, LLC, Valhalla, NY (US)

(72) Inventors: Theodore L. GERSTLE, Valhalla, NY (US); Christopher GERSTLE, Valhalla, NY (US)

(21) Appl. No.: 14/723,437

(22) Filed: May 27, 2015

Related U.S. Application Data
(60) Provisional application No. 62/003,473, filed on May 27, 2014.

(51) Int. Cl. B29C 67/00; A61F 2/50; G05B 15/02; G06F 19/00

(52) U.S. Cl. CPC B29C 67/0088; G06F 19/321; A61F 2/5046; G05B 15/02; B33Y 50/02

(57) ABSTRACT

The present disclosure provides a database and methods to allow users to download and/or upload scanned or raw data for objects from or into a database. The data relating to the objects will be stored in a design file on the database, and will allow for end users to print the objects on a 3D printer at their location. A fee may be charged by a manager of the database. The database will allow for sharing of images between health care providers so that they can reproduce and/or redesign the saved images to produce instruments, cutting guides, models, prosthesis for procedures that are specifically suited for their procedure and patient.

### CROSS-REFERENCE TO RELATED APPLICATIONS

The present application claims the benefit of U.S. Provisional Application Ser. No. 62/003,473, filed on May 27, 2014, which is incorporated herein by reference.

### BACKGROUND OF THE DISCLOSURE

1. Field of the Disclosure

The present disclosure relates to a database and methods for accessing the same. More particularly, the database of the present disclosure will allow users to access, input, and download models, renderings, or files relating to many different types of medical devices and tools that can be downloaded, revised, and/or printed.

2. Description of the Related Art

Three-dimensional (3D) printing in medical applications is currently costly, labor intensive, and narrowly limited to only a few applications. Most importantly, 3D printing with current devices and methods take between 24 hours and 30 days to produce a device capable of helping a patient. Current 3D printing devices or methods may utilize a procedure by which a doctor wishing to develop a prosthetic (such as a replacement organ or bone) takes an image of the relevant area or part of the patient's body, and then sends the image off to a remote site to create the product. After days to weeks, the product that arrives is often a poor fit and may require a second production cycle, leading to a further delay in treatment, and in many cases, a second surgery. Additional surgeries can present a host of complications and dangers to the patient, as well as a significant amount of discomfort and emotional distress, as the surgical wound site will often necessitate remaining open between surgeries. The present disclosure provides devices and methods for overcoming these deficiencies.

### SUMMARY OF THE DISCLOSURE

The present disclosure provides a database and process for producing prostheses, tools, instruments, guides, and models for a wide variety medical procedures (e.g., surgery) and/or teaching. Designs for the prostheses et al. can be uploaded to the database and reproduced on a three-dimensional (3D) manufacturing platform. In one embodiment, the customer can be required to pay a fee in order to print the design at their location. The database will store all designs uploaded to it by the end-user for future use at a fee to all users. This provides convenience, flexibility, and mobility for surgical application that are not currently available.

For ease of description, the term “prosthesis” is used in the present disclosure to refer to the types of implants, bone replacements, tissue replacements, prostheses, or even whole organs that can be designed and created in the devices and methods of the present disclosure. Thus, the term “prosthesis” as used in the present disclosure may refer to, without limitation, customized facial implants (bony, airway, vascular, or soft tissue implantation), facial fractures and repair, microtia framework, ocular, vascular, and cardiac prostheses, nasal prostheses, maxillary prostheses, palatal prostheses, septal prostheses, cranial vault prostheses, mandibular bone replacement (bone graft printout), maxillary bone replacement, customized soft tissue implant (all areas of the body including but not limited to airway stents, vascular stent or graft, or percutaneous or surgical vascular occlusion devices.), hand/extremity implants/prostheses, joint replacement (e.g., small joints of the wrist/fingers), large joint replacement (e.g., hips, knees, shoulder), spine corpus replacement, long bone replacement (femur, tibia, fibula, radius, ulna, humerus), rib cage replacements, pelvic defect repairs, large joint replacements, non-implantable prosthetics (e.g., fingers, other appendages, limbs, orthotics, splints, or facial obturators), combinations thereof, or other suitable implants.

The terms “instruments” and “tools” are used to denote devices that are useful to surgeons or technicians in medical procedures. Suitable but not limiting examples can be scalpels or retractors.

By “intra-operative use”, the present disclosure means that the prosthesis or instrument is printed or fabricated within the same operative procedure or in the same operative location as the location where the image on which the prosthesis is based is acquired. Current devices or methods may refer to “rapid-prototyping”, but this typically means that when the image of a specific part is acquired, it is then sent off to be printed remotely, in a process that may take several weeks. With use of the terms “ultra-rapid prototyping” and “intra-operative”, the present disclosure distinguishes between these processes. In the method of the present disclosure, the required prosthesis can be provided during the surgical procedure. This may all optionally take place while the patient is under a single anesthetic, as discussed in greater detail below.

The term “file” is used herein for ease of description, and denotes a file, image, design, or other digital data relating to a prosthesis or medical tool that is stored in the database of the present disclosure. For example, a “file” may signify an image relating to a new scalpel designed by a user, and the associated image data. A file may also be a three-dimensional rendering of a prosthesis, for example for a bone of the orbital cavity of a patient. A file of the present disclosure may be in any format suitable for digital storage, manipulation, and transfer to a printer. Suitable examples are, but are not limited to, .CAD, .DWG, .STL, .OBJ, or .thing files. As will be discussed in greater detail below, the user can upload or download the file, and send the file to a printer at an end-user station. Again, the files can be directed to prostheses, tools, instruments, guides, models, or any other suitable devices for medical applications.

Thus, in one embodiment, the present disclosure provides a process for producing a prosthesis or surgical tool by accessing a database, comprising the steps of: uploading a file to the database; displaying the file on a display device; transmitting of the image to a manufacturing platform; and printing the prosthesis or surgical tool according to the file on the manufacturing platform.

In another embodiment, the present disclosure provides a method of producing a surgical implement or prosthesis, comprising the steps of: accessing a database; accessing a file on the database, wherein the file contains information relating to the surgical implement or prosthesis; downloading the file; transmitting the file to a printer; and printing the surgical implement or prosthesis using the file.

### BRIEF DESCRIPTION OF THE DRAWINGS

FIG. 1 shows a flowchart describing the way data can be uploaded to the database of the present disclosure, and shared with other users for an end user fee.

### DETAILED DESCRIPTION OF THE DISCLOSURE

The present disclosure provides a database and marketplace, residing on a server maintained by an administrator or in the cloud, that stores one or more files relating to medical tools, equipment, and/or prostheses. The database and marketplace will allow for ultra-rapid prototyping of prosthetics and instruments for a variety of medical applications. A user (e.g., a doctor, technician, or teacher) can download a file from the database and marketplace and send it to a printer so that the depicted object can be printed. The user can also store the file locally for review and manipulation, or conduct such review and manipulation while the file is in the database. The user can also upload an image or file to the database and marketplace. As discussed in greater detail below, the file that the user can upload to the database can be design specifications for an instrument, model, cutting guide or prosthesis. The user can also use multiple imaging or mapping techniques to create the image or file, and then upload it to the database.

The file can then be printed at the user's location. In another embodiment, the file that the user uploads can be manipulated or reviewed by other users at different locations. In one embodiment, the user, as the customer, would pay a service fee to the entity managing the database for either printing or uploading the file. The user will also be able to search a wide range of other files relating to other devices that can be printed at the user request, again at times for a fee. Also, the user will be able to manipulate files already in the database, to customize them for the user's particular project. The new, manipulated files or image can be saved to the database for future printing. The database may also allow for open information-sharing, thus allowing surgeons and physicians to have access to other inventors' works, while allowing a level of customization to tailor the product to their specific patient.

Thus, the database and marketplace (hereinafter “database” for ease of description) of the present disclosure mitigates or eliminates many of the problems with current medical device printing systems. With the present database, a user can access a library of potentially suitable files relating to prostheses or medical tools and print the necessary equipment right at their location. The user can also manipulate stored files to desired or specific needs for their application or procedure. This saves a tremendous amount of time and is greatly beneficial for the patient, as there are no longer significant lead times for ordering custom tools or prostheses. With the database of the present disclosure, a surgical procedure requiring custom prostheses or implements can be conducted while the patient is under a single anesthetic. This has tremendous benefits for the surgeon and patient alike.

The database of the present disclosure can also be suitable for various collaborative uses. For example, in a joint research project, one or more members of the group could create and upload a file for review, comment, and modification by the other members of the group. The image may be password protected so that only members of a specific group can access it. In another application, a person or business entity desiring to raise venture capital may use the database of the present disclosure to showcase an idea for investors. Investors could browse the database for ideas of projects or companies to invest in. In another application, a company could sponsor or host a space on the database, and allow customers or potential research partners to upload files for possible collaborative or joint ventures.

The database is also beneficial for teaching applications. Such flexibility and convenience is not available in current systems. The database of the present disclosure will overcome doctors' or technicians' inability to have devices that fit their exact specifications for medical procedures, which can be printed on-site even during surgery.

In addition, with the database of the present disclosure, doctors, medical instructors, or other users in the field can upload files relating to new ideas for new surgical treatments, tools, prostheses, or other suitable medical information to the database. In this way, the database of the present disclosure can function as an online or cloud-based research platform. Users would have the ability to research and collaborate with other users of the database to develop ideas relating to new medical devices and procedures. The database could serve in this fashion as an open source for medical advancements. Potential inventors could review the database and marketplace for investment opportunities on new concepts that the inventors may not have the time or means to develop themselves. Many inventors decide not to pursue commercialization of their ideas due to the prohibitive cost involved. The database of the present disclosure would enable them to upload their ideas for a wider audience, and if someone else wishes to pursue the idea further, they can negotiate with the owner. Companies in the business of manufacturing medical devices could also occupy a branded space on the database, to sell their products. Searchers conducting patentability or freedom to operate searches on behalf of patent applicants could use the database and market place as a prior art database. Users could be required to sign non-disclosure agreements before reviewing the concepts located in the database and marketplace.

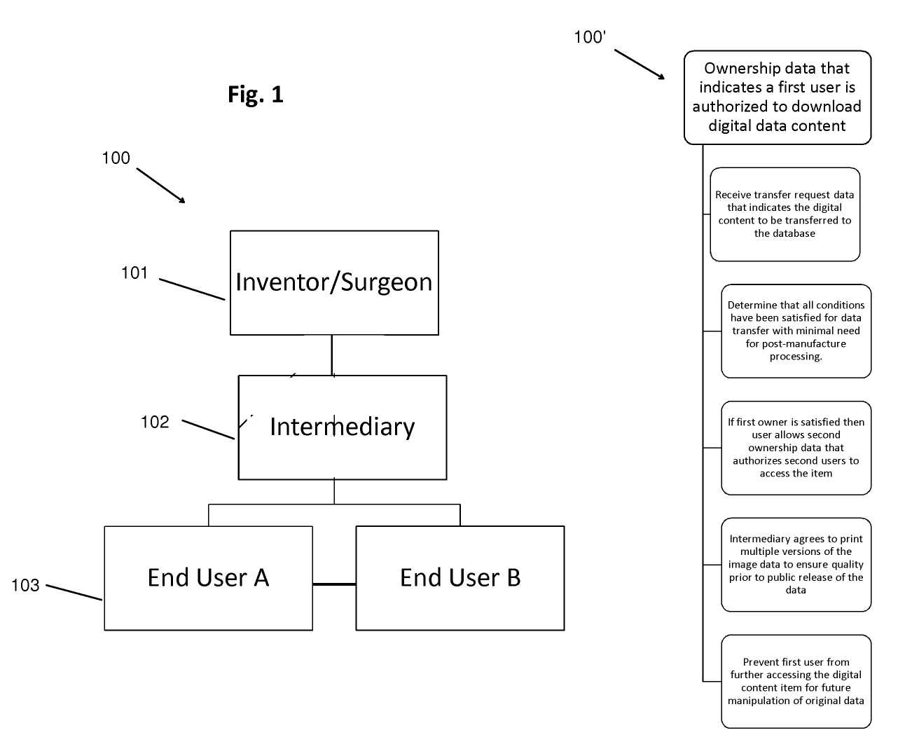

Referring to FIG. 1, a flow chart illustrating one possible application 100 for the database of the present application is shown. A surgeon (i.e, an inventor) has a specific patient need for a prosthesis or tool that is either too costly with current offerings from vendors, or altogether does not exist, and creates a design or file new prosthesis or tool (101). An intermediary helps with the design, manufacture of that prosthesis or tool and at no cost to the inventor, saves the design in a searchable database (102). When the need arises in another application in the future, it can be downloaded and used on another machine by other users (103) to design, alter, and manufacture it elsewhere in the world. The original inventor then receives a royalty for the that use and all future uses. Each time the design is altered, the original inventor receives a royalty along with the last “editor” of the design. Process (100′), also shown in FIG. 1, details the steps that can take place within the database during application (100).

With the database of the present disclosure, high-quality customized prostheses, instruments and prostheses can be designed on-site. Users are able to take the original designs and make them customized to their patient-specific or site-specific needs. These modifications or customizations can be minimal or significant. The database will also allow physicians to print the right tool for the job quickly as they can search the database for an instrument. That instrument can be readily printed for them on site and during a single anesthetic. The database manager can provide quality assurance by iterative printing to ensure high quality products are available to the end users. The database manager can test print all uploaded images to the database to ensure the quality of the image uploads for other end users.

Users can also upload their customized designs after completion. This can be very beneficial when a team of medical staff, for example, is collaborating on a particular procedure. When a customized design is uploaded to the database, multiple users can access the design and modify it if needed. The database will only accept designs for upload once it has met all conditions to ensure high quality printing with only minor post-manufacture processing (PMP).

The devices and methods of the present disclosure are discussed in the context of three-dimensional (3D) printing (also known as “additive manufacturing”). 3D printing may include, but is not limited to, such methods as fused deposition modeling, fused filament fabrication, robocasting, electron beam freeform fabrication, direct metal laser sintering, electron beam melting, selective laser melting, selective heat sintering, selective laser sintering, plaster-based 3D printing, laminated object manufacturing, stereolithography, and digital light processing. Processes of “subtractive” manufacturing may be employed as well. In this embodiment, the image acquisition device would send an image of a desired prosthesis to the computer, as described above. The final image, with or without modification, is sent to a fabricator. The fabricator uses subtractive methods to produce the prosthesis, where the prosthesis can be hewn from a solid piece of implantable material. The subtractive methods may include lathing the prosthesis, cutting with laser-, water-, or air-blade-cutting tools, stamping, grinding, or carving.

According to the present disclosure, during a surgical procedure a doctor or technician can acquire a file relating to a surgical tool, or find the correct device in the database. Software working in conjunction with the database can display the image and allow the user/surgeon to modify the file as necessary, or as dictated by the patient's anatomy. The software program can allow the doctor, a technician, with or without input from the patient themselves, to customize the scanned image to desired settings or features. The final image, customized as applicable, is then sent to a printer or fabricator for creation. As previously discussed, the manufacturing process occurs at the surgeon's location, despite the fact that technical design characteristics have been accomplished elsewhere/previously. These customized designs can be input into the database so they can be reused, or another instrument or prosthesis can be made from the customized scan with minimal changes. The design file, as available from the database or after customization by the user, can either be downloaded and stored locally at the user's location, or sent directly to the printer.

With the devices and methods of the present disclosure, a user can find the correct instrument or prosthesis from the files on the database, and retool the data to their specifications if necessary. The reworked file can then be sent to a manufacturing platform, and designed object can be created. The total period time for this process—from file acquisition, retooling of the file if necessary, and printing—can be from five minutes to twenty-four hours, or any subranges therebetween. The period of time can also be from thirty minutes to twelve hours, or any subranges therebetween. These periods of time allow for the surgery to be completed while the patient is still under anesthesia. This is also known as “intra-operative use”.

By “intra-operative use”, the present disclosure means that the prosthesis and/or instrument is printed or fabricated within the same operative procedure (i.e., under a “single aesthetic”) or in the same operative location as the location where the image on which the prosthesis is based is acquired. Currently available devices or methods may refer to “rapid-prototyping”, but this typically means that when the image of a specific part is acquired, it is then sent off to be printed remotely, in a process that may take several weeks. With use of the terms “ultra-rapid prototyping” and “intra-operative”, the present disclosure distinguishes over these processes. In the method of the present disclosure, the required prosthesis can be provided during the surgical procedure. One of the most unique aspects of this disclosure is that the scanning of the patient and processing of the image as well as printing of the prosthetic or other implantable devices for the patient can be done under a single anesthetic.

The printer or fabricator of the present disclosure can also eliminate the time associated with sterilization of an implantable prosthesis in currently available devices and methods. In currently available systems, when the doctor or surgeon receives an implantable prosthesis after a long printing delay, there is additional time associated with sterilization of the prosthesis, which further adds to the cost of the procedure and risk for the patient. With the devices and methods of the present disclosure, however, this time is significantly reduced or eliminated completely. The printer or fabricator can provide an already-sterilized prosthesis for immediate use. In the case of a prosthesis produced via computer-guided lathe, the machining of the prosthesis will still likely still require sterilization, but the lathing process can be more expeditious than printing, so the additional time for sterilization should not be prohibitive from a safety stance.

The materials suitable for the prostheses of the present disclosure may vary. The materials can include polylactic acid and acrylonitrile butadiene styrene, which are approved by the United States Food and Drug Administration for implantable devices. Other materials contemplated may include rubber, ceramics, light-cured polymers, metals, and implantable antibiotic-impregnated solids.

In addition to providing suitable prostheses and tools for use with patients, the database and methods of the present disclosure can provide surgical planning models for the doctor and patient. A doctor can search the database for a model to show the patient or scan one into the database and have it printed. This will decrease the number of office visits a patient would need prior to surgery and allow for a single surgery as any scannable object can be printed by these methods and made ready for surgery quickly and safely.

The database of the present disclosure can be used in conjunction with other co-located three-dimensional scanning and printing devices. The term “co-located” in this context means that the scanner and printer are located in the same facility or even in the same room, to allow for ultra-rapid prototype scanning and printing. These include such systems that may be in a traditional permanent hospital or medical facility, or more mobile modular units that can be transported. In either case, a computer or scanner associated with the three-dimensional systems can access the database through a communications network. Thus, a user could scan a patient or tool, and upload a file to the database of the present disclosure. The file can then be used or manipulated as described above.

While the present disclosure has been described with reference to one or more particular embodiments, it will be understood by those skilled in the art that various changes may be made and equivalents may be substituted for elements thereof without departing from the scope thereof. In addition, many modifications may be made to adapt a particular situation or material to the teachings of the disclosure without departing from the scope thereof. Therefore, it is intended that the disclosure not be limited to the particular embodiment(s) disclosed as the best mode contemplated for carrying out this disclosure.

1. A process for producing a prosthesis or surgical tool by accessing a database, comprising the steps of:
uploading a file to the database;
displaying the file on a display device;
transmitting of the image to a manufacturing platform; and
printing the prosthesis or surgical tool according to said file on said manufacturing platform.

2. The process of claim 1, further comprising the step of charging a user of said database for said printing step.

3. The process of claim 1, further comprising the step of allowing a user to manipulate said file on said display device, between said displaying and said transmitting steps.

4. The process of claim 1, wherein said display device and said manufacturing platform are disposed at the same location.

5. The process of claim 1, wherein a database manager reviews said file after said uploading step, and compares it to pre-existing quality standards.

6. The process of claim 1, wherein a period of time between said transmitting step and said printing step is between 10 minutes and twenty-four hours.

7. A method of performing a surgical procedure on a patient, employing the process of claim 1, wherein the method comprises the steps of:
using said prosthesis or surgical tool during the surgical procedure.

8. The method of claim 7, comprising the step of employing all of the process steps of claim 1 while the patient is under a single anesthetic.

9. A method of producing a surgical implement or prosthesis, comprising the steps of:
accessing a database;
accessing a file on said database, wherein the file contains information relating to the surgical implement or prosthesis;
downloading said file;
transmitting said file to a printer; and
printing the surgical implement or prosthesis using said file.

10. The method of claim 9, further comprising the step of customizing or modifying said design file before said downloading or said transmitting step, thereby creating a customized file.

11. The method of claim 1, further comprising the step of uploading said customized design file to said database.

12. The method of claim 9, further comprising the step of paying a fee to a manager of said database before said downloading step.

13. A method of performing a surgical procedure on a patient, employing the process of claim 9, wherein the method comprises the step of:
using said prosthesis or surgical tool during the surgical procedure; and

[symbol]

14. The method of claim 13, comprising the step of employing all of the process steps of claim 9 while the patient is under a single anesthetic.
